(19) United States
(12) Patent Application Publication
(10) Pub. No.: US 2016/0193733 A1
(43) Pub. Date: Jul. 7, 2016

(54) ROBOT FOR MEDICAL ASSISTANCE

(71) Applicant: Fatemah A.J.A. Abdullah, Sharg (KW)

(72) Inventor: Fatemah A.J.A. Abdullah, Sharg (KW)

(21) Appl. No.: 15/066,778

(22) Filed: Mar. 10, 2016

Related U.S. Application Data
(63) Continuation-in-part of application No. 14/227,188, filed on Mar. 27, 2014, now abandoned.

(51) Int. Cl. B25J 11/00; G06F 19/00; A61B 5/01; A61B 5/021; A61B 5/00; A61J 7/04; A61B 34/30

(52) U.S. Cl. CPC B25J 11/009; A61J 7/0409; A61J 7/0481; A61J 7/0418; G06F 19/3462; A61B 34/30; A61B 5/021; A61B 5/4833; A61B 5/01; A61B 5/743; Y10S 901/01; A61B 2560/0214

(57) ABSTRACT

A robot for providing medical assistance, delivery medications and testing materials to a patient is disclosed. The robot includes a set of wheels and a mechanism for bringing the robot to a patient as well as a mechanism for avoiding objects and individuals. A computer and program provides initial guidance and instructions for medications and testing and medical history of a patient. The computer programs the robot to distribute medications, perform and record test results and in an emergency to telephone a healthcare provider. The robot also includes a transmitter and receiver for sending pictures, test results and medical history to a remote station. Further the robot includes a storage battery, a charger and means for connecting the charger to a source of electricity.

### CROSS REFERENCE TO RELATED APPLICATIONS

The present application is a Continuation-In-Part of U.S. patent application Ser. No. 14/227,188, filed on Mar. 27, 2014, and priority is hereby claimed under 35 U.S.C. §120 based on this application and is hereby incorporated by reference in its entirety into the present application.

### FIELD OF THE INVENTION

This invention relates to a robot for providing medical assistance to a patient and more particularly to a robot for providing medical assistance, medication, testing, reminders and alerts.

### BACKGROUND FOR THE INVENTION

The use of robots to complement medical care has been known for a number of years. For example, a U.S. Patent of Y. Wang, U.S. Pat. No. 6,925,357 discloses a Medical Tele-Robotic System. As disclosed therein a robotic system that includes a remote controlled robot includes a camera, a monitor and a holonomic platform all attached to a robot housing. The robot may be controlled by a remote controlled station that also has a camera and a monitor. The remote control station may be linked to a base station that is wirelessly coupled to the robot. The cameras and monitors allow a care giver at the remote location to monitor and care for a patient through the robot. The holonomic platform allows the robot to move about a home or facility to locate and follow a patient.

A more recent U.S. Patent of Koselka, U.S. Pat. No. 7,228,203 discloses an Autonomous Personal Service Robot. The patent discloses a robot to monitor a patient for symptoms of distress and provide assistance. The system may include sensors to detect situations before they affect people such as smoke, heat, temperature and carbon monoxide. The system can provide security for the home and may comprise features such as a medicine dispenser and blood pressure cuff. Features such as broadband internet, MP3 player, reading lights and eye glass tracker provide butler type capabilities that enable the system to appeal to markets beyond the elderly and infirmed. The system may also include an X10 transmitter/receiver to automatically control various household lights and appliances. The system also includes a robot arm that enables the robot to fetch items, turn on and off wall switches and open a refrigerator.

Still further, a U.S. Patent of Ziegler, U.S. Pat. No. 8,195,333 discloses a Companion Robot For Personal Interaction. As disclosed a mobile robot interacts with a human resident and performs a room-traversing search procedure prior to interacting with the resident and may verbally query whether the resident is present. Upon finding the resident, the mobile robot may facilitate a teleconferencing session with a remote third party, or interact with the resident in a number of ways. For example, the robot may carry on a dialogue with the resident, reinforce compliance with medication or other schedules, etc. In addition, the robot incorporates safety features for preventing collisions with the resident and the robot may audibly announce and/or visibly indicate its presence in order to avoid becoming a dangerous obstacle.

Notwithstanding the above, it is presently believed that there is a need and a potential commercial market for an improved robot for medical assistance. There should be a commercial market for such devices because they provide test apparatus for blood pressure, temperature and blood sugar. Further the robot is designed to provide medication over a seven day period as well as instructions for taking doses at proper times. In addition to the above, the robot in accordance with the present invention provides a refrigerated compartment for insulin or the like and means for automatically modifying a family member or caregiver that a scheduled dosage has not been acknowledged by the patient.

### BRIEF SUMMARY OF THE INVENTION

In essence the present invention contemplates a robot for providing medical assistance, medications, testing, reminders and alerts and comprises or consists of the following:

A housing, a set of wheels and means for moving the housing from a storage area to a first destination in the proximity of a patient and wherein the means for moving the robot include an electric motor and gear train as well as a storage battery and connection for recharging the battery.

The robot also includes means for avoiding collisions with objects or individuals and may include a physical sensor for a more sophisticated system based on a radar sensor. For example, the robot in accordance with a preferred embodiment of the invention includes a collision avoidance subsystem, as illustrated in FIG. 9. The subsystem includes an infrared sensor that upon sensing an object in its pathway sounds an alert and moves to avoid a problem. However, when no obstruction is sensed the robot proceeds and continues to search for obstructions.

A computer having data storage, a time schedule for taking each medication over a seven day period and a patient's medical history stored on an insertable card so that it may be removed and taken with the patient to a hospital or remote location. Still further the robot includes a first air tight storage device for containing a seven day supply of medicines and a refrigerated storage compartment for storing a seven day supply of insulin or other medication. This may include a plurality of alcohol swabs or other disinfectants, a pen including the medication and a plurality of needles one for each individual injection.

Further the robot in accordance with the preferred embodiment of the invention includes remote means for testing a patient's temperature, and recording the temperature, day and time of reading. In addition to the above, means are provided for testing a patient's blood pressure and means for testing a patient's blood sugar are provided as well as means for recording the test results and day, and each time of day of such tests. Still further the system includes means for acknowledging taking medications and means for reminding a patient to take the medication if unacknowledged after a preselected period as for example 45 minutes as well as means after a second time delay for automatically telephoning a relative or healthcare provider. Further the robot in accordance with the preferred embodiment of the invention includes remote means for testing a patient's temperature, and recording the temperature, day and time of reading. In addition to the above, means are provided for testing a patient's blood pressure and means for testing a patient's blood sugar are provided as well as means for recording the test results and day, and each time of day of such tests. The way that the robot uses to test the blood sugar is by using a Gluco Track DF-F Noninvasive Glucose Meter. After that the robot records the test results. Still further the system includes means for acknowledging taking medications and means for reminding a patient to take the medication if unacknowledged after a preselected period as for example 45 minutes as well as means after a second time delay for automatically telephoning a relative or a healthcare provider.

The system also includes a screen for indicating the lists of medication, times last taken, patient's temperature, blood pressure, blood sugar, times of scheduled testing as well as the patient's medical history and contact information for contacting a patient's personal physician. Still further, the system includes means for alerting a patient at the end of the day of any medical appointments for the next day. Finally, the robot includes means for connecting a battery charger and means connecting the system to the battery charger as well as a warning light for indicating a low charge and means actuateable by said robot for returning the robot to a charging site and coupling the charger and the robot to a source of electricity for charging the battery.

Finally, the system includes means for a patient to remotely contact the robot and bring the robot to the proximity of the patient. For the insulin injection, the robot will have the supply needles, pens and disinfectant and the patient himself injects the insulin.

The invention will now be described in connection with the accompanying drawings wherein like reference numerals have been used to identify like parts.

### DESCRIPTION OF THE DRAWINGS

FIG. 1 is a schematic illustration on one side of a robot in accordance with the present invention;

FIG. 2 is a schematic illustration along an opposite side of the robot shown in FIG. 1;

FIG. 3 is a schematic illustration of a weekly medicine regime with a dispenser that provides dosages scheduled for two times a day. These medications are carried by the robot in a first compartment and may be merely brought to the individual or include a mechanism for removing the medication from the compartment and bring it to an elevated position for ready access by the patient;

FIG. 4 is a schematic illustration of a remote temperature sensor as used in connection with the present invention;

FIG. 5 is a schematic illustration of a blood pressure testing apparatus as used in the present invention;

FIG. 6 is a schematic illustration of a non-invasive glucose meter for testing blood sugar in connection with the robot as shown in FIGS. 1 and 2;

FIG. 7 is a flowchart illustrating the drive mechanism, programmed guidance system for the robot in accordance with the invention;

FIG. 8 is a block diagram of an electrical system for the robot in accordance with the invention;

FIG. 9 is a schematic block diagram of an IR physical sensor as used in the invention;

FIG. 10 is a schematic illustration showing a medical assistant robot for providing medical assistance, medications, testing supplies and tests as well as alerts that include taking a patient's blood pressure. The system or device also includes a blood pressure cuff and a mechanical linkage to raise the cuff above and over the side of a bed and a flexible connection for applying the cuff to an upper arm of a patient;

FIG. 11 is a schematic illustration of a front side of a medical assistant robot illustrating a first set of sensors for sensing a human body in the robot's pathway; and

FIG. 12 is a schematic illustration of a rear side of a medical assistant robot illustrating a second set of sensors for sensing a human body in the robot's pathway assuming the robot is in a reverse mode.

### DETAILED DESCRIPTION OF THE PREFERRED EMBODIMENTS OF THE INVENTION

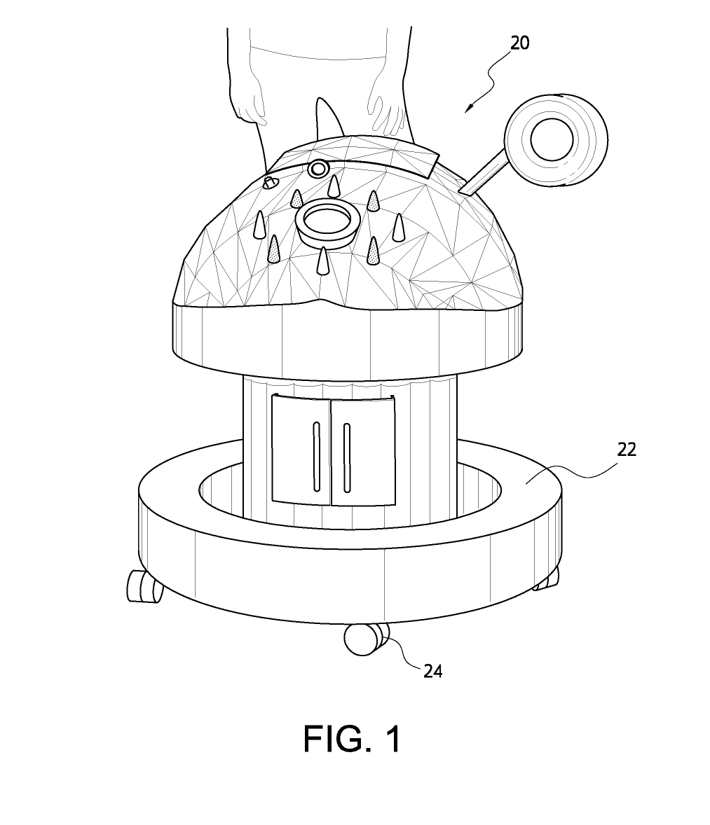

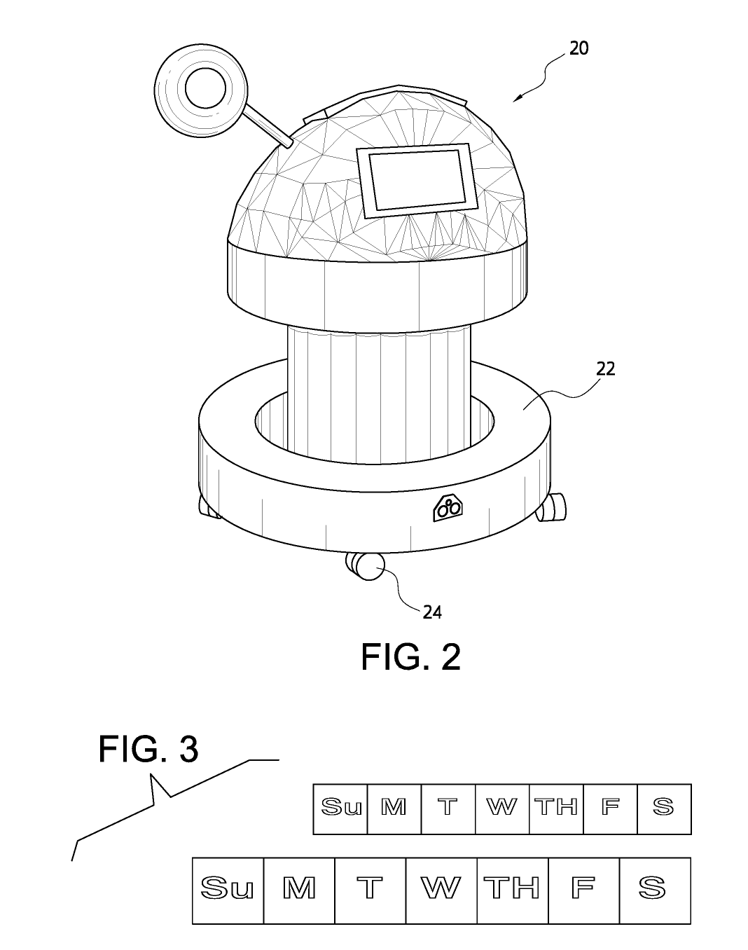

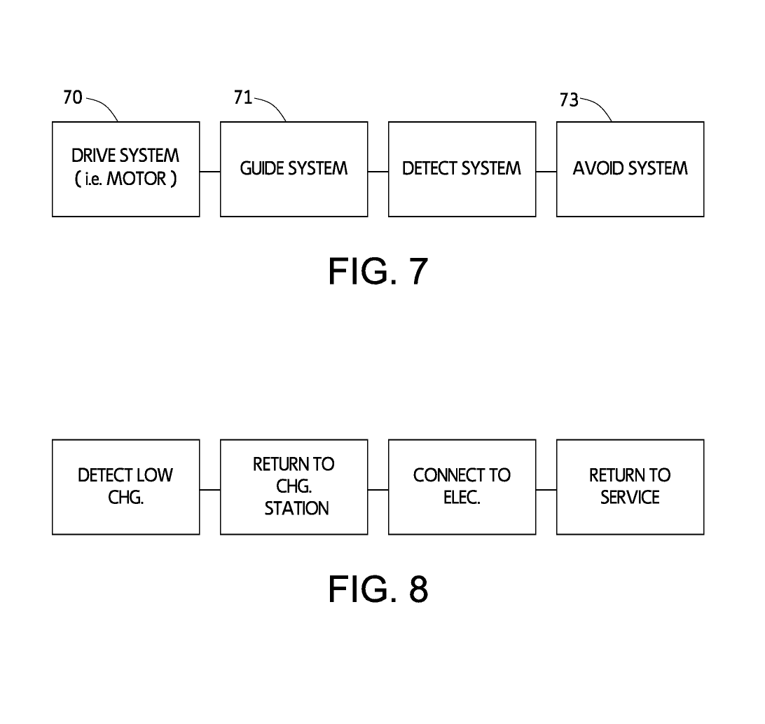

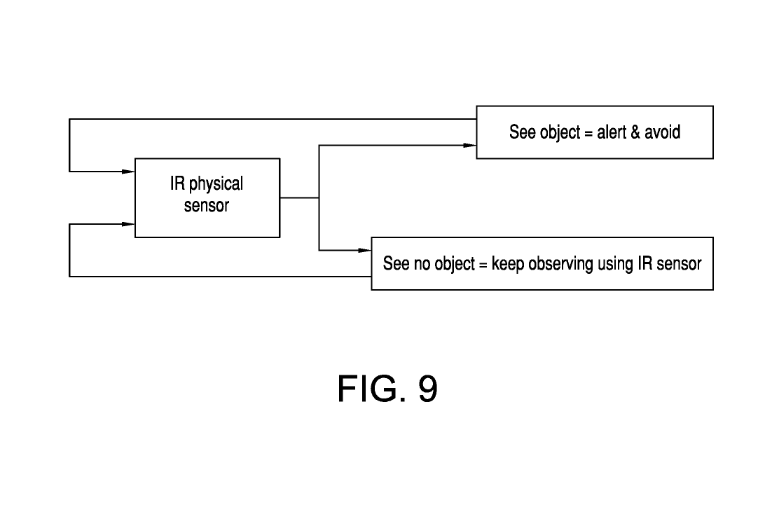

As illustrated in FIGS. 1 and 2, a robot 20 for providing medical assistance, a seven day supply of oral and/or injectable medications, testing supplies and testing facilities as well as a computer for recording test results, medical records as well as notices of appointments and medical alerts. As shown, the robot 20 includes a base 22, a set of wheels 24 and a drive mechanism 70 (see FIG. 7), for transporting the robot from a storage area to a patient and back.

The drive mechanism 70 includes an electric motor 71, a gear train connected to a guide system 71 for guiding the robot 20 from a storage area to a patient. The system 70 also includes a system to detect objects in its pathway in order to bypass those objects in accordance with an avoidance system 73. The robot 20 includes a self-contained charger that is activated by plugging it into a source of electricity. The robot 20 contains a sound emitting warning unit and speaker to warn individuals of a moving robot in the area. The sound system is similar to those used when a large truck is in reverse or when an electrical vehicle is moving about an area inhabited by individuals. The robot also includes a sensor or a detector system including a sensor and means for avoiding objects and individuals by moving around such objects or individuals. This avoidance system of the robot is conventional as incorporated in a Sear's Robot Scooba 340 Vacuum as well as other brands of robot vacuum cleaners. In a preferred embodiment of the invention, the robot 20 includes a sensor for detecting individual humans in its general pathway, avoiding them and/or increasing the sound level of the warning to warn individuals of a moving robot in the area.

With respect to testing an individual's blood sugar the robot 20 includes a small opening 31 as a schematic illustration of a typical testing for an individual. As disclosed, the testing is accomplished in one of two ways. In an initial approach, the robot 20 delivers the test supplies as for example an alcohol swab, lancet and lance for drawing blood from a patient's finger. A test strip for interaction with blood drawn up by a test strip and a meter that provides a blood sugar reading. This is a typical procedure practiced by many if not most diabetics in the United States and in many foreign countries. With this approach the readings are entered in a reader or other conventional means as for example a small keyboard connected to a reader that is shown on the screen 21 of the robot 20.

Today there is an alternative approach. In this approach a Gluco Track DF-F Noninvasive Glucose Meter (Israel) is provided. The meter consists of a small sensor that is clipped to an earlobe and connected to a handheld control and display unit. The problem with past non-evasive glucose meters is that few if any gave accurate results under varying conditions. To overcome this problem, the Gluco Track instrument employs three methods simultaneously utilizing ultrasound, electromagnetic and thermal technologies to obtain blood glucose readings. The results are then correlated and averaged and the results displayed on a LCD screen. It is anticipated that the results will be displayed on the computer monitor with the time and date of a test stored in memory for review by a physician or medical caregiver in an emergency situation.

The robot 20 also includes a blood pressure monitor device such as an Omron 10 Series BP785, an upper arm blood pressure monitor that includes a ComFit cuff that is easy to apply to one's own arm. The system uses a positioning icon to signal whether it is applied correctly. Other models manufactured by other manufactures such as a Panasonic EW3109W or the like may be used as well as wrist blood pressure models. Notwithstanding the make or model it is preferred to setup a testing apparatus to display the results on a monitor or screen 30 and record the results and time and date of the test in the computer memory.

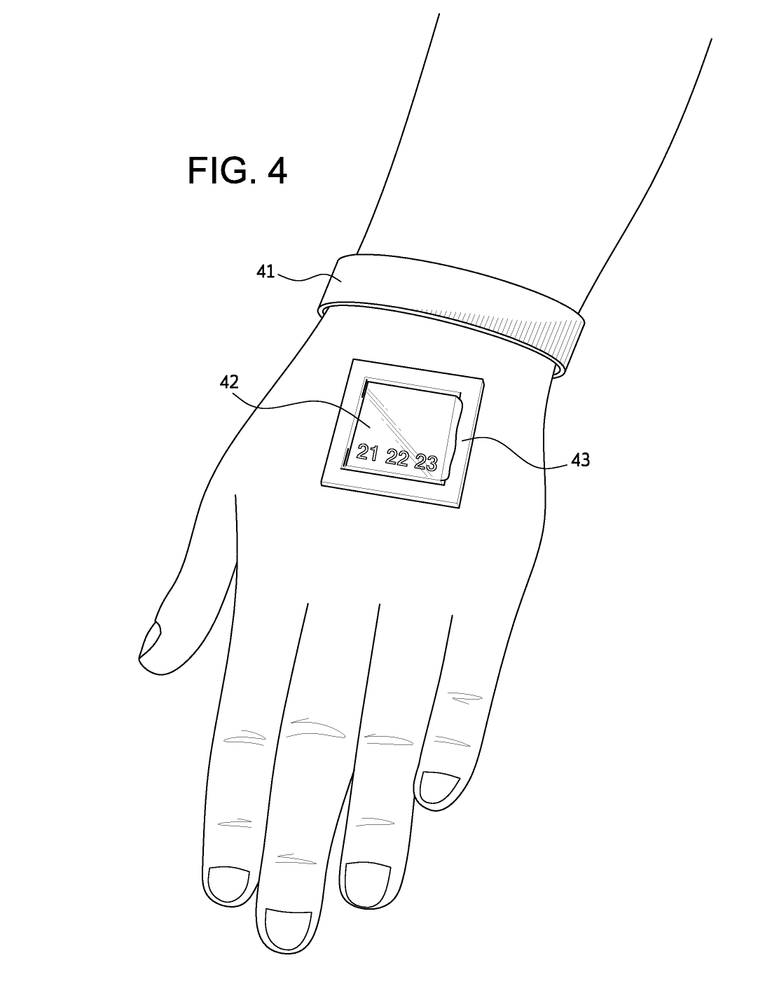

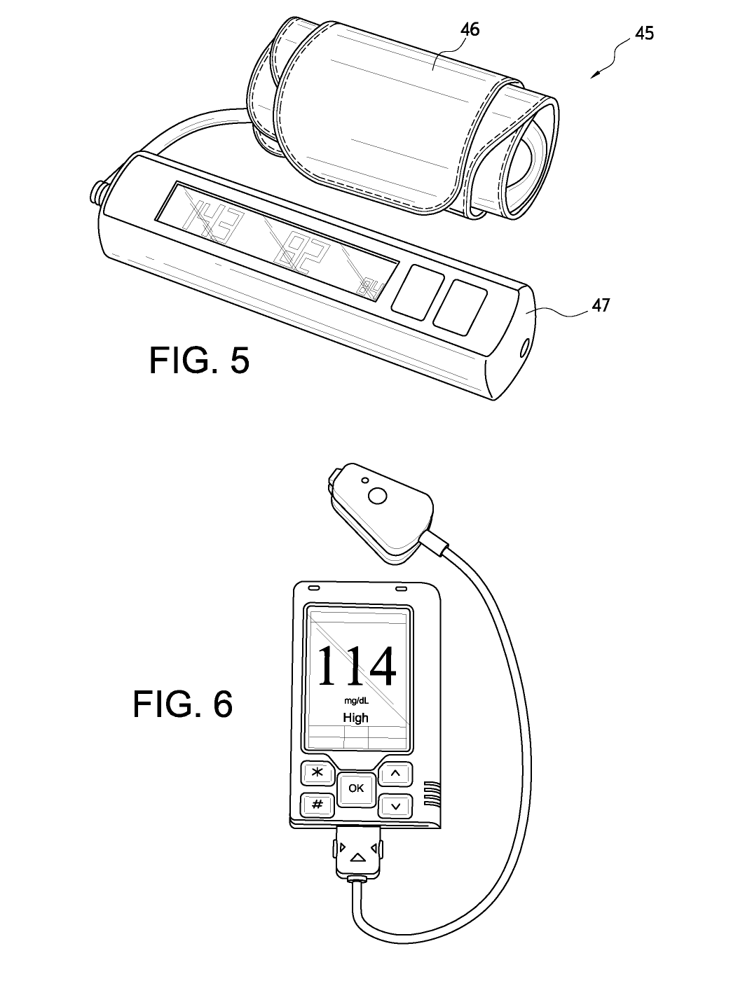

A further feature of the robot 20 resides in a remote temperature that measures the patient's body temperature. As illustrated in FIG. 4, the patient wears a bracelet 41 and a patch 42 that is applied to the back of a patient's hand by a piece of double sided tape. The patch measures a patient's body temperature using a liquid crystal thermometer 43 that is held in place with a piece of double sided tape. A glass slide and a thin glass cover and a temperature monitor as illustrated. The temperature is illustrated in F.° and/or C°. The bracelet communicates with the robot's computer via blue tooth or the like via a transmitter in the bracelet.

A blood pressure monitor 45 is illustrated in FIG. 6 and as shown is an upper arm unit that includes a ComFit cuff 46 and a monitor 47 that displays a patient's blood pressure. Such monitors are manufactured by Omron healthcare.com. Similar units are manufactured by Panasonic and others that can be used in place of the Omron device. Wrist blood pressure monitors are also available and is assumed modifiable to indicate the blood pressure on a monitor as for example screen shown in the robot and stored in memory with the time and date of the series of tests.

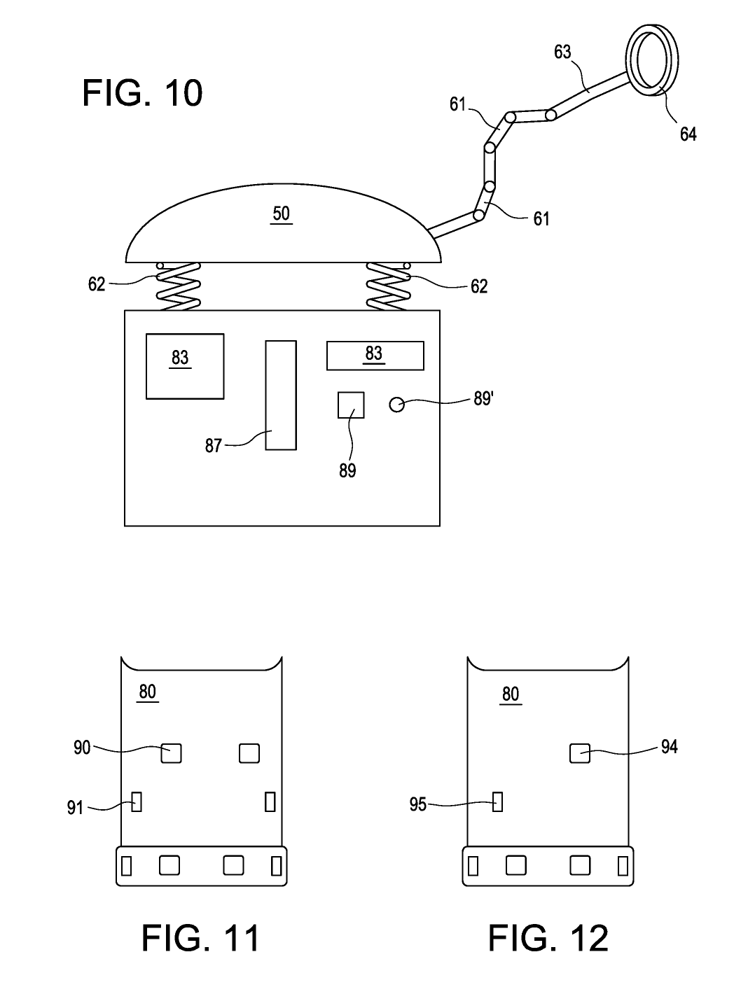

As shown in FIG. 10 a robot head 50 or an upper portion thereof is adapted to be moved upwardly above the body of the robot by a pair of scissor-like links or mechanical mechanism 62 such as screws or pneumatic or hydraulic pistons. In this way, an upper arm encircling cuff 64 is elevated up above the side of a bed (not shown) and partially across the bed so that a patient can test their blood pressure. In addition to the mechanical linkage 61, a conventional flexible connector 63 allows a patient sufficient room to allow the patient to wrap the cuff around an upper portion of their arm between their elbow and shoulder. Then after an increase in air pressure increased by the patient, the patient can test their blood pressure in a conventional manner

As shown in FIG. 10, an embodiment of the invention includes a first compartment 83 for containing a supply of a week-long supply of medication as illustrated in subsequent drawings as for example a supply of morning medications, a second supply of midday medications and a final supply of dinnertime medications and perhaps a still further supply of evening medications to be taken before bedtime. The robot also includes a second refrigerated compartment 85 for insulin and other refrigeration dependent medications. Test equipment is stored in a separate compartment 87. Further, a 24-hour clock 89 and print activator prints the time when various medications are taken by pressing a button 89 on robot 81 or remote module (not shown).

With respect to FIG. 11, a first or front side of the robot 80 includes a first pair of physical or human body sensors detected in the robot's forward pathway. For example, an infrared sensor/detector 90 and ultrasonic detector 91 detects a physical or human presence in the robot's pathway. As illustrated in FIG. 12, an infrared sensor/detector 94, ultrasonic detector/sensor 95 and computer program (not shown) avoids collision with objects and individuals each of which include an infrared and an ultrasonic sensor.

With respect to FIG. 12, the rear portion of the robot 80 further includes a second set of sensor detectors including an infrared sensor 90 and an ultrasonic detector 91 detects a physical or human body in the robot's pathway if the robot is traversing in a reverse mode.

While the invention has been described in connection with the above-identified preferred embodiments, it should be recognized and understood that changes and modifications may be made therein without departing from the scope of the appended claims.

What is claimed is:

1. A robot for providing medical assistance, medications, testing supplies and tests and alerts, said robot consisting of:
a housing, a set of wheels, and a drive mechanism for moving said housing from a storage area to a first destination in the proximity of a patient and wherein said drive mechanism for moving said housing includes an electric motor, gear train and a storage battery;
a pair of guide systems including a first of said pair of guide systems disposed in a forward portion of said robot for avoiding collisions with objects and/or human individuals in a general forward pathway and a second of said pair of guide systems disposed in a rear portion of said robot for avoiding collision with objects and/or human individuals in a rearward pathway and wherein each of said pair of guide systems include an infrared sensor/detector and an ultrasonic sensor/detector and increasing the sound level of a warning to warn individuals of a moving robot;
a computer having data storage, a timer for taking medications over a seven day period and a patient's medical history stored on an insertable card so that it may be removed and taken with the patient to a hospital or remote location and said robot includes:
a first airtight storage device for containing a seven day supply of medication and a refrigerated storage compartment for containing a seven day supply of insulin or other medications, a plurality of alcohol swabs or other disinfectants, a pen including insulin or other medications requiring refrigeration and a plurality of needles one for each individual injection and a separate compartment for test apparatus and supplies;
a remote thermometer for testing a patient's temperature, and a 24 hour clock and an actuator for recording the temperature, day and time of reading and wherein the patient wears a bracelet and a patch applied to the back of a patient's hand by a piece of double-sided tape and the patch measures the patient's body temperature using a liquid crystal thermometer that is held in place with a piece of double-sided tape:
a blood pressure monitor device for testing a patient's blood pressure including a blood pressure cuff, mechanical linkage and flexible connection to raise said cuff above a side of a bed and partially over the patient to allow the patient to apply said cuff to an upper portion of the patient's arm, and a positioning icon to signal that it is correctly applied and to display the results on a monitor or screen;
a blood sugar monitor for testing a patient's blood sugar and means for recording the test results and day and time of day of each test, wherein the testing is accomplished by delivering the test supplies including an alcohol swab and lancet for drawing blood from a patient's finger, a test strip for interaction with drawn blood and a meter providing a blood sugar reading;
means for acknowledging taking medication and means for reminding a patient to take the medication if unacknowledged after 30-45 minutes and means after a second time delay for telephoning a relative or healthcare provider of an emergency situation;
a screen for indicating the list of medications, time last taken, patient's temperature, blood pressure, blood sugar time of testing, patient's medical history and contact information for patient's personal physician;
an alert mechanism for alerting a patient at the end of the day of any medical appointments for the next day;
a battery charger, a warning light for low charge and means activatable by said robot for coupling said robot to said charger or a source of electric energy for charging said battery; and
means for a patient to remotely contact said robot and bring said robot to the proximity of the patient.
